(19) United States
(12) Patent Application Publication
(10) Pub. No.: US 2008/0087288 A1
(43) Pub. Date: Apr. 17, 2008

(54) APPARATUS AND METHODS FOR EFFECTING THERAPY ON A BODY

(75) Inventor: Jeffrey WUN, Fremont, CA (US)

Correspondence Address: LEVINE BAGADE HAN LLP, 2483 EAST BAYSHORE ROAD, SUITE 100, PALO ALTO, CA, 94303, US

(73) Assignee: Kairos Systems, Inc., Fremont, CA (US)

(21) Appl. No.: 11/548,654

(22) Filed: Oct. 11, 2006

(51) Int. Cl. A61N 2/02; A61H 23/00; A61H 9/00; A61N 1/18

(52) U.S. Cl. 128/898; 600/14; 601/112; 601/15; 607/104; 607/3; 607/62

(57) ABSTRACT

Apparatus and methods for effecting therapy on a body using an electric/magnetic or an electromagnetic field is disclosed herein generally having a housing defining a reservoir for receiving a portion of the body at least partially within an electrically conductive medium. A portion of the reservoir contains at least one positively charged electrode and at least one negative charged electrode secured to the housing by a retaining member. The apparatus may also comprise of a magnet in proximity to the electrodes. The electrodes induce a static or a fluctuating electric field which is regulated by a controller unit such that the electric field is perpendicular to the magnetic field to impart a resulting force which may act upon the user's body to effect therapy. The electric current may be further regulated by measuring a physiological parameter and correlating the electric and/or magnetic field strength according to the measured physiological parameter.

### BACKGROUND OF THE INVENTION

The present invention relates to apparatus and methods for effecting therapy on a portion of a patient's body. More particularly, the present invention relates to apparatus and methods for improving circulation using an electric field and a magnetic field, (e.g. a static or pulsed magnetic field, a static or pulsed electromagnetic field, etc) while measuring a physiological parameter of the body. For example, exposing a part of the body to an electric field between heartbeats will typically create an electromotive force which may cause positive and negative ions in blood to move in the opposite direction, depending on the magnetic pole encountered and the charge of the ion, potentially increasing the flow of blood.

The issue of pain treatment is an extremely urgent health problem. Pain is often accompanied with or results from poor circulation. Poor circulation can occur in any area of the body. However, poor circulation predominantly affects the extremities (peripheries) of the body such as the fingers, hands, feet and/or ankles. Poor circulation in the extremities may result in the body's inability to heal injuries (i.e. sores, infections, cuts, etc.). In many cases injuries do not heal at all without special care. The reason for this is, in part, that the blood carries vital elements (such as oxygen) that the body's tissues need for vitality and healing.

Additional symptoms of poor blood circulation may include neuropathy, coldness, tingling, burning and numbness in the feet or hands, shortness of breath, low energy, irregular heart beats, sluggish memory, and/or lack of stamina. Poor blood circulation can contribute to the formation of diabetes, arthritis, high LDL cholesterol, high blood pressure, angina, and/or heart disease.

Severe cases of poor circulation may be treated with medication, surgery or minimally invasive interventional procedures. However, because of the various effects on the body, many conventional products exist on the market to assist with increasing blood circulation. Traditional massage and/or heat devices exist, such as foot spas or baths and mechanical massage chairs, ottomans, or pads. However, these devices typically do not penetrate the surface of the skin and therefore are not as effective as exposure to magnetic fields. There are also devices on the market that utilize magnetic fields, such as pads and wands. However, these devices lack biofeedback regulation and/or do not provide additional relaxation features that make compliance and effective use favorable.

Accordingly, there is a need for apparatus and methods that increase the circulation of blood while regulated by biofeedback as well as optionally providing additional relaxation features such as bubbles, mechanical movement, and/or heat.

### BRIEF SUMMARY OF THE INVENTION

An apparatus and method for effecting therapy on a portion of a patient's body may generally comprise, in one variation, a housing defining a reservoir for receiving a portion of the body at least partially within an electrically conductive medium. The electrically conductive medium may be a fluid such as water. Additionally, a portion of the reservoir may contain at least one positively charged electrode and at least one negative charged electrode. The electrodes may be conductive metal plates which typically are aligned parallel to one another.

In addition to the reservoir, the housing may also have a magnet in proximity to the electrodes which may be secured to the housing by a retaining member that may be in electrical communication with the electrodes. The apparatus may further comprise a power source and a controller unit in electrical communication with the electrodes and/or magnet. The controller unit may be configured to regulate an electric current to the electrodes (via the retaining member) such that an electric field is induced through the electrically conductive medium in the presence of a magnetic field generated via the magnet. In yet another variation, the controller unit contains a current limiting switch and optionally may also contain a timer.

In another variation, the electric field that is generated may be static while in another variation, the electric field may be configured to fluctuate in amplitude and/or frequency. Furthermore, the magnet may be an electromagnet adapted for generating an electromagnetic field and may be in electrical communication with the controller unit. In another variation, the magnetic field generated via the electromagnet may be static or it may be configured to fluctuate.

Moreover, the apparatus and its variations may further comprise a biofeedback unit worn or otherwise in proximity to the user such that the unit is adapted to detect a physiological parameter from the patient's body and which is in communication, wired or wirelessly, with the controller unit. The biofeedback unit may be adapted to detect any number of physical parameters, e.g., heartbeat, blood pressure, etc., such that the detected parameters is utilized in regulating an operation of the device to optimize its effect in treating the body. The apparatus may additionally comprise a user interface in communication with the controller unit to optionally display the physiological parameter. In one method of use, the controller unit may be adapted to regulate an electric current through the electrodes such that an electric field generated via the electrodes through the conductive medium is induced between heartbeats.

The housing may comprise a retaining wall and a floor surface for containing the conductive medium. The floor of the apparatus may be smooth or it may comprise of a plurality of protrusions to facilitate a massaging effect on the body, such as the feet. The apparatus may also further comprise a variety of additional mechanical features. For instance, the apparatus may further comprise a vibrating mechanism adapted to vibrate the floor of the reservoir in which case the vibrating mechanism may comprise a motor having an output shaft and a rotating member eccentrically coupled to the housing. Additionally, the apparatus may further comprise one or more heating elements in thermally conductive contact with the conductive medium. One variation of the heating element may utilize a fluid channel in fluid communication with the reservoir and a pump which circulates the fluid through the fluid channel and into thermal contact with the heating element to heat the temperature of the fluid.

Another variation of the apparatus may include a bubble generator within the housing to urge air through one or more channels into the fluid such that bubbles are formed and vented through a plurality of openings into the fluid.

### BRIEF DESCRIPTION OF THE SEVERAL VIEWS OF THE DRAWINGS

FIG. 1 schematically illustrates the resulting force generated by an electric current placed in a magnetic field.

FIG. 2 illustrates the force generated by an electric current place in a magnetic field on a portion, such as the foot, of a patient's body.

FIGS. 3A-3B show side and top views, respectively, of a variation of a housing containing a positively charged and a negatively charged electrode which induce an electric field in proximity to a magnetic field.

FIGS. 4A-4B show side and top view, respectively, of another variation comprising of a plurality of positively and negatively charged electrodes.

FIG. 5 is a graph schematically illustrating a constant electric field.

FIG. 6 is a graph schematically illustrating a fluctuating electric field.

FIG. 7 illustrates a biofeedback unit in communication with a controller unit.

FIG. 8 shows a biofeedback unit secured to a wrist of a user in communication with a controller unit.

FIG. 9 illustrates a biofeedback unit in wireless communication with a controller unit.

FIG. 10 is a graph schematically illustrating a static electric field induced between heartbeats.

FIG. 11 is a graph schematically illustrating a fluctuation electric field induced between heartbeats.

FIG. 12 shows a user interface in communication with a controller unit.

FIG. 13 illustrates a user interface in wireless communication with a controller unit.

FIG. 14 shows a side view of a variation of a housing containing a positively charged and a negatively charged electrode for inducing an electric field in proximity to a magnetic field generated by an electromagnet.

FIG. 15 shows a side view of another variation of a housing containing a plurality of protrusions and a vibrating mechanism.

FIG. 16 shows a top view of another variation of a housing containing a heating element and a bubble generator.

### DETAILED DESCRIPTION OF THE INVENTION

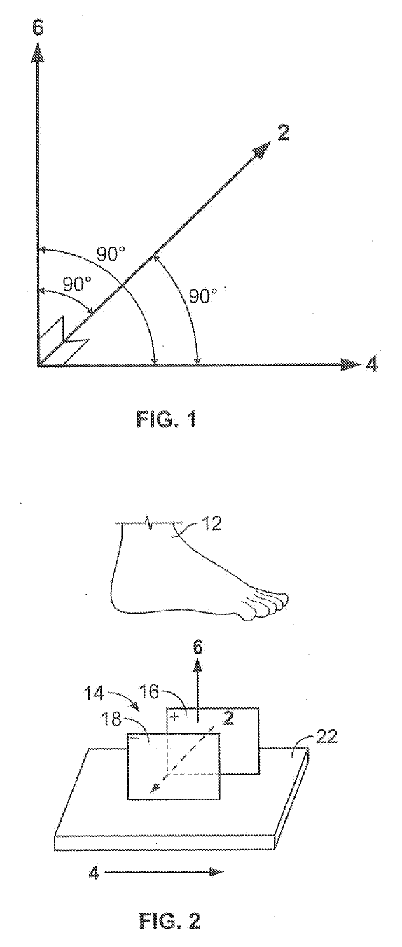

Blood, like all tissues, contains electrically charged ions. A physics principle known as Faraday's Law states that a magnetic field will exert a force on a moving ionic current. Furthermore, as shown in FIGS. 1-2, an extension of Faraday's law called the Hall effect states that when a magnetic field 2 is placed perpendicular to the direction of flow of an electric current 4, it will create a perpendicular force 6 which will tend to deflect and separate the charged ions. While the deflection of ions will be in opposite directions depending on the magnetic pole encountered and the charge of the ion, this force 6 is not based on the attraction or repulsion of like and unlike charges.

The Hall effect implies that when a magnet is placed over flowing blood in which ionic charges (such as Na+ and Cl−) exist, some force 6 will be exerted on the ions. Furthermore, the separation of ionic charges will produce an electromotive force, which is a voltage between points in a circuit. In theory, this produces a very small amount of heat. These physical effects account for the purported effects of magnetic field therapy. For example, when a magnetic field 2 with alternating north and south poles is placed over a blood vessel, the influence of the field 2 will cause positive and negative ions (for example, Na+ and Cl−) to bounce back and forth between the sides of the vessel, creating flow currents in the moving blood not unlike those in a river. The combination of the electromotive force, altered ionic pattern, and the currents causes blood vessel dilation with a corresponding increase in blood flow. This effect and its corresponding increase in blood flow may be accomplished using the apparatus and methods described below.

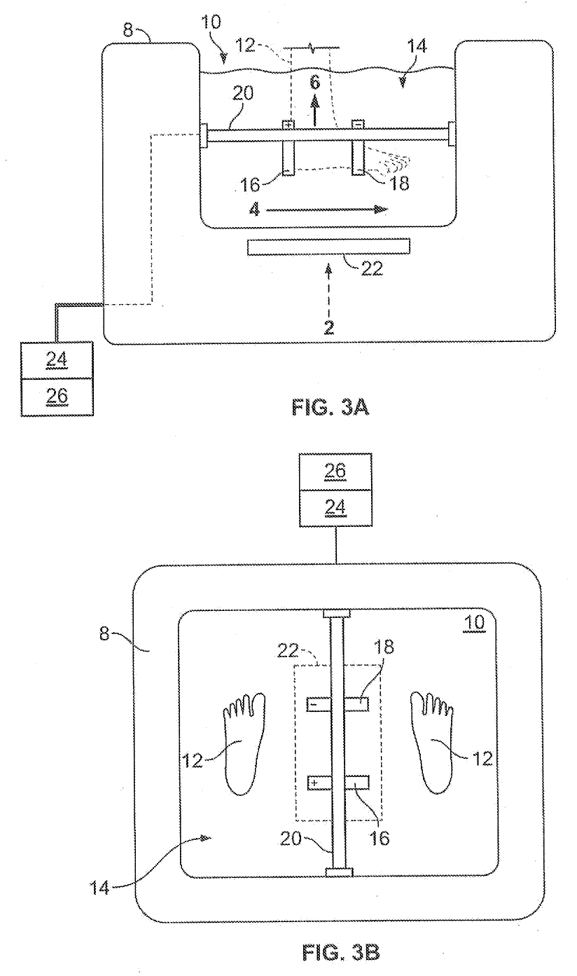

FIGS. 3A-3B illustrate an apparatus for improving circulation. The apparatus may typically comprise a housing 8 defining a reservoir 10 for receiving a portion of the body 12, such as a foot or feet of the user. The housing 8 may be made from plastic or other nonporous, nonconductive material and the reservoir 10 may contain an electrically conductive medium 14, e.g., a fluid such as water or a gel. With the reservoir 10 filled, the user's foot or feet 12 may be at least partially immersed within the medium 14. Although the foot or feet are described and illustrated, this is intended to be illustrative and other portions of the user's body such as the hands, arms, etc., may be immersed alternatively.

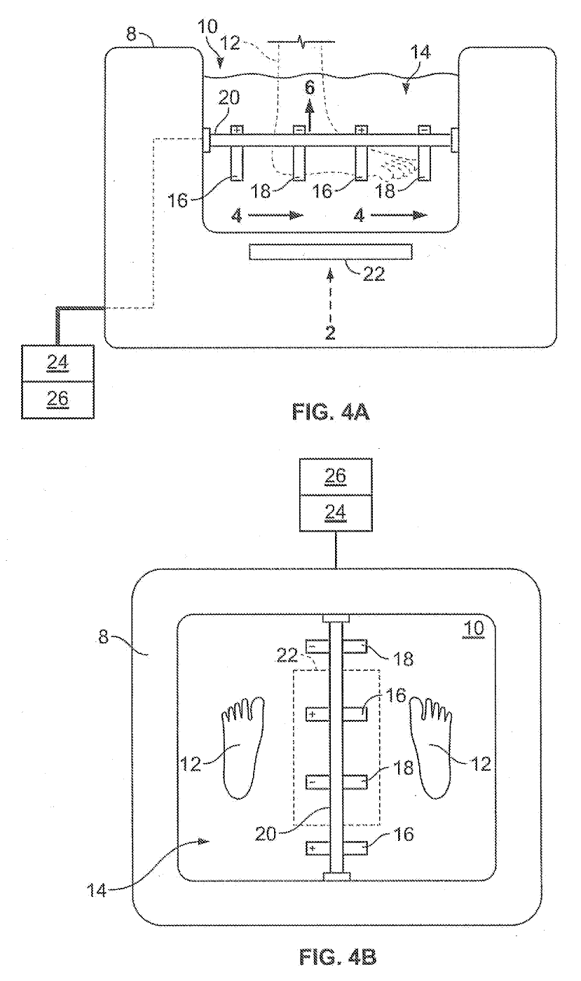

At least one positively charged electrode 16 and at least one negatively charged electrode 18 may be contained within the reservoir 10 while in other variations, as shown in FIGS. 4A-4B, a plurality of positively charged electrodes 16 and negatively charged electrodes 18 may be provided. The electrodes may each be formed as a conductive metal plate positioned parallel relative to one another such that each surface of the plate is positioned in apposition relative to one another. However, the electrodes may be fabricated from any number of conductive materials (e.g., stainless steel) and positioned in a manner that optimally allows for an electric current 4 to be passed therebetween.

The electrodes may be secured to the housing 8 by a retaining member 20 which may be positioned within or along the housing 8. The retaining member 20 may also be securable to the housing 8 and in electrical communication with the electrodes. Accordingly, the retaining member 20 may be made of an electrically conductive material (e.g., stainless steel, Nickel, Platinum, etc.). In other alternatives, the retaining member 20 may also be made of a nonconductive material (e.g., plastic) whereby the retaining member 20 may include an electrically conductive material, e.g., placed through a lumen in member 20, to allow for electrical communication with the electrodes. While the retaining member 20 may be permanently affixed to the housing 8, it may also be removable from and/or movable within the housing 8.

The apparatus may also comprise at least one magnet 22 in proximity to the electrodes. Furthermore, the apparatus may additionally include a controller unit 24 in wired or wireless communication with the electrodes and/or magnet 22. In one variation, the controller unit 24 may be external to or integrated within the housing 8. Additionally, the apparatus may further comprise a power source 26, which may also be contained within the controller unit 24 or external to the unit, in electrical communication with the electrodes.

To effect a portion of a patient's body 12 at least partially immersed within a electrically conductive medium 14, the controller unit 24 may be configured to regulate an electric current 4 to the electrodes such that an electric field is induced through the electrically conductive medium 14 in the presence of and/or proximity to a magnetic field 2 generated via the magnet 22. The electric field may be induced by passing an electric current 4 from the positively charged electrode 16 to the negatively charged electrode 18 which are at least partially immersed in the conductive medium 14. The electric field may be induced perpendicularly relative to the magnetic field 2.

The resulting force 6 effected on the patient's body may be equal to or less than 0.5 N, although the resulting force may be regulated to yield a greater force than 0.5 N. To avoid overexposure to the patient's body 12 to excess electrical current, the controller unit 24 may have a current limiting switch and it may also have a timer to regulate or limit the time of exposure of the electric and magnetic fields to a portion of a patient's body. Typically, exposure may last anywhere between 20-45 minutes. However, shorter or longer exposure periods may also be effective.

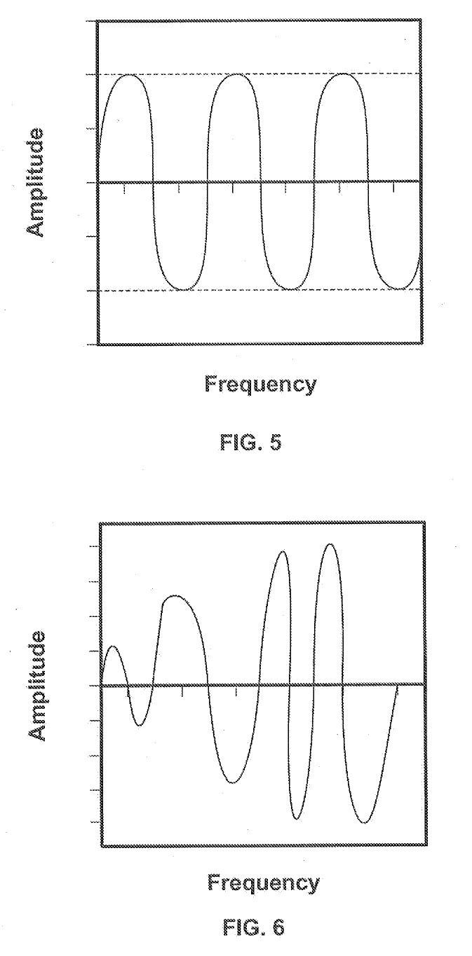

The controller unit 24 may regulate the electric current 4 so that the electric field may be configured to be a static electric field. As shown in FIG. 5, an electric current 4 pulsed at a constant amplitude and a constant frequency may induce a constant electric field. Alternatively, the controller unit 24 may regulate the electric current 4 so that the electric field may be configured to be a fluctuating electric field. As shown in FIG. 6, an electric current 4 pulsed at a varied amplitude and/or a varied frequency may induce a fluctuating electric field. Varying the amplitude and/or the frequency of the electric current 4 may also assist with increasing the flow of blood in areas of the body that may be subject to various conditions, such as atherosclerosis. The frequency of the pulses may be up to 1200 Hz and the amplitude may be between 18-26 V, although different frequencies and amplitudes may be used. The number of pulses may range anywhere from one to four or more.

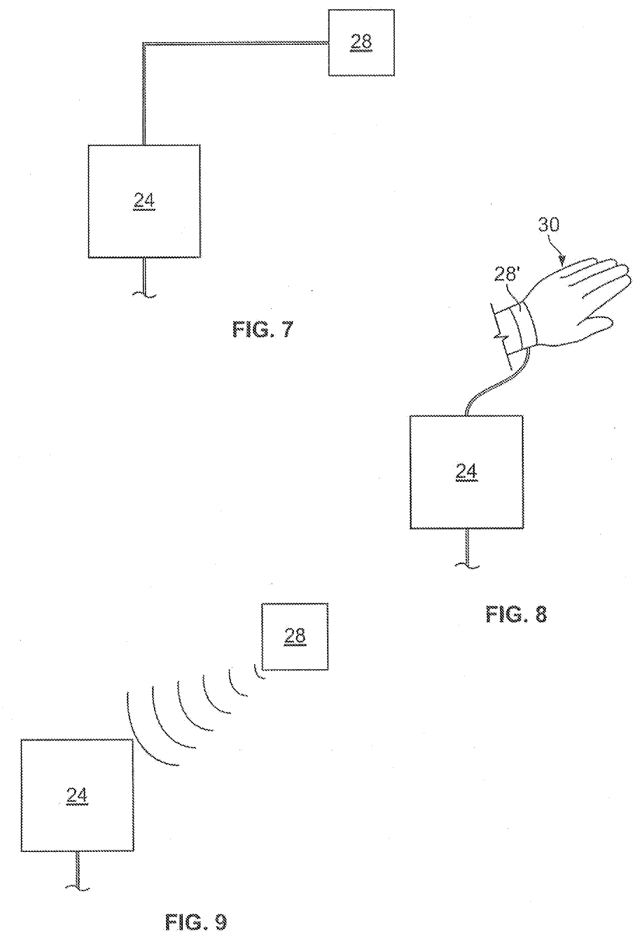

Any magnetic forces generated by a field affecting fluid movement in blood vessels would have to overcome both the normal, pressure-driven turbulent flow of blood propelled by the heart and the normal thermal-induced Brownian movement of the particles suspended in the blood. This may be accomplished by measuring a physiological parameter of the body while the body portion is subjected to the electric and magnetic field 2. The physiological parameter may be measured using a biofeedback unit 28 in wired or wireless communication with the controller unit 24 as shown in FIGS. 7-9. The biofeedback unit 28 may be worn directly on the user's body or it may be held in proximity to the body to detect any number of physiological parameters from the patient's body, including, but not limited to, heartbeat, blood pressure, and/or cardiac cycle, etc. FIG. 8 illustrates a biofeedback unit 28′ secured to the wrist 30 of a patient such that a heartbeat (i.e. pulse) may be detected and communicated to the controller unit 24. However, the biofeedback unit 28 may be securable to any portion of the body which is able to provide a physiological parameter. The biofeedback unit 28 may communicate with the controller unit 24 via a wired connection or it may be configured to communicate wirelessly, as shown in FIG. 9. Accordingly, the biofeedback unit 28 may be equipped with a transmitter and the controller unit 24 may be equipped with a receiver adapted to receive a wireless signal from the biofeedback unit 28, e.g. via infrared.

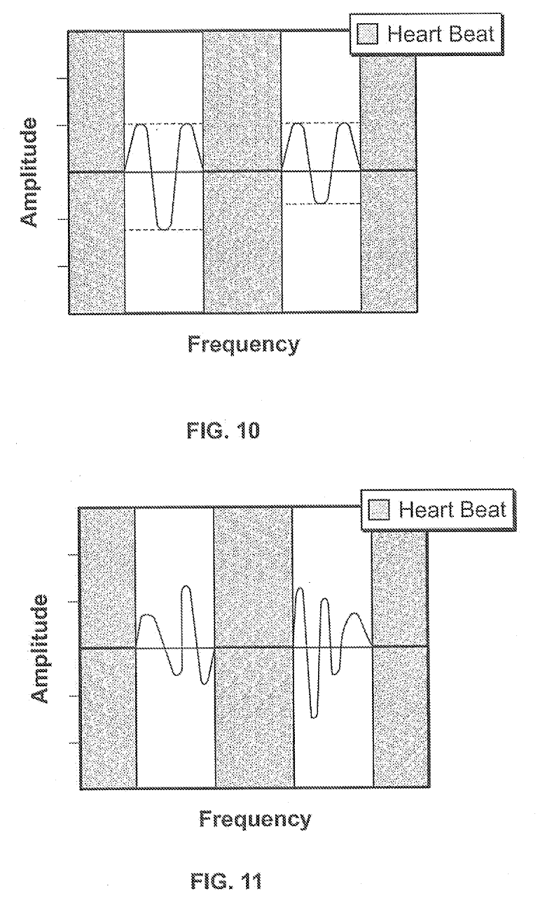

The controller unit 24 may regulate the electric current 4 through the electrodes such that an electric field generated via the electrodes through the conductive medium 14 is induced between heartbeats or in some corresponding manner depending upon the physiological parameter measured or detected. The controller unit 24 may further regulate the electric current 4 by pulsing the electric current 4 in a corresponding manner between detected heartbeats at a constant amplitude and a constant frequency, as shown in FIG. 10. Alternatively, the controller unit 24 may further regulate the electric current 4 by pulsing the electric current 4 in a corresponding manner between detected heartbeats at a varied amplitude and a varied frequency, as shown in FIG. 11. In the event the biofeedback unit 28 fails to detect and/or measure a physiological parameter, the controller unit 24 may regulate the electric current 4 by pulsing the electric current 4 at a default value of 72 pulses per minute, or any other value depending upon the desired result.

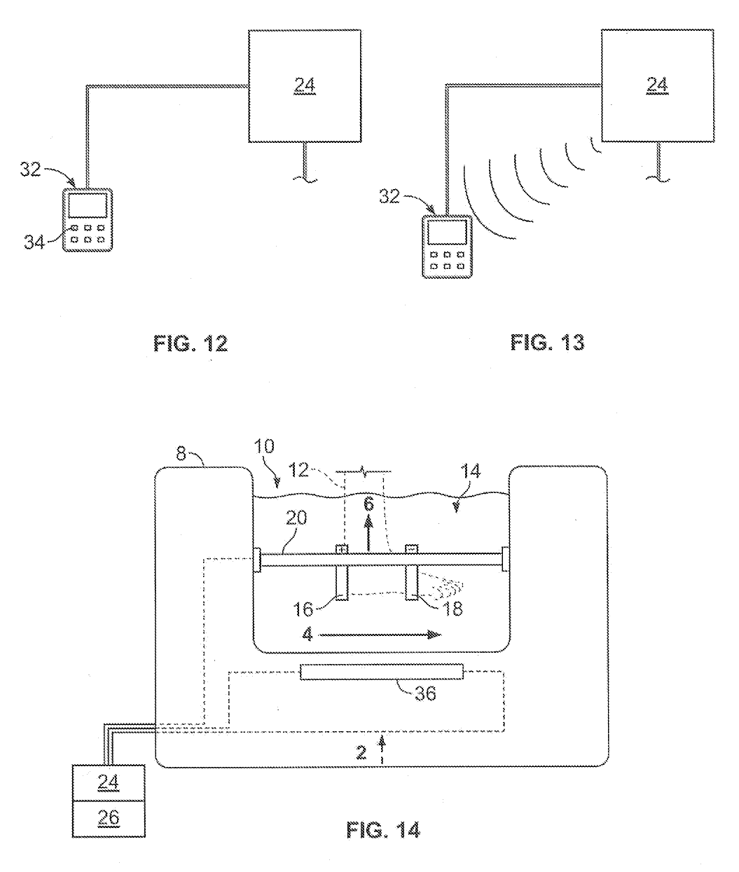

The apparatus may further comprise a user interface 32 to display the physiological parameter as illustrated in FIG. 12. The user interface 32 may have one or more features 34, such as buttons, which allow the patient to manually control the apparatus. For example, the patient may use the user interface to turn the apparatus ON/OFF or control the intensity of the generated electric and/or magnetic field or to control the timer feature. The user interface 32 may be in communication with the controller unit 24 to program the controller or to receive feedback for display to the user. In one variation, the user interface 32 may be integrated with the controller unit 24 or it may be separated from the controller unit 24. Alternatively, the user interface 32 and the controller unit 24 may be configured to communicate wirelessly as shown in FIG. 13 in which case the user interface 32 may be equipped with a transmitter and the controller unit 24 may be equipped with a receiver to receive a wireless signal from the user interface 32, e.g., infrared, Bluetooth, etc.

The magnet 22 may be a ferrous magnet or alternatively an electromagnet 36 which generates an electromagnetic field in electrical communication with the controller unit 24, as shown in FIG. 14. An electromagnet 34, in one variation, is a magnet having a coil of insulated wire wrapped around a soft iron core that is magnetized only when current flows through the wire. Variations of the apparatus having a magnet 22 and methods described herein for the electric field also apply to the apparatus having an electromagnet 36 and methods for the electric field. In addition, the magnetic field 2 generated by the electromagnet 36 may be static in the same manner as the electric field may be static. Alternatively, the magnetic field 2 generated by the electromagnet 36 may fluctuate in the same manner as the electric field may fluctuate.

In one variation, the controller unit 24 may regulate the electric current 4 through the electromagnet 6 so that a static magnetic field is generated and the controller unit 24 may also regulate the electric current 4 to the electrodes through the electrically conductive medium 14 so that a static electric field is generated. In another variation, the controller unit 24 may regulate the electric current 4 through the electromagnet 6 so that a static magnetic field is generated and the controller unit 24 may also regulate the electric current 4 to the electrodes through the electrically conductive medium 14 so that a fluctuating electric field is generated. In yet another variation, the controller unit 24 may regulate the electric current 4 through the electromagnet 6 whereby a fluctuating magnetic field is generated and the controller unit 24 may also regulate the electric current 4 to the electrodes through the electrically conductive medium 14 so that a static electric field is generated. In another variation, the controller unit 24 may regulate the electric current 4 through the electromagnet 6 so that a fluctuating magnetic field is generated and the controller unit 24 may also regulate the electric current 4 to the electrodes through the electrically conductive medium 14 so that a fluctuating electric field is generated.

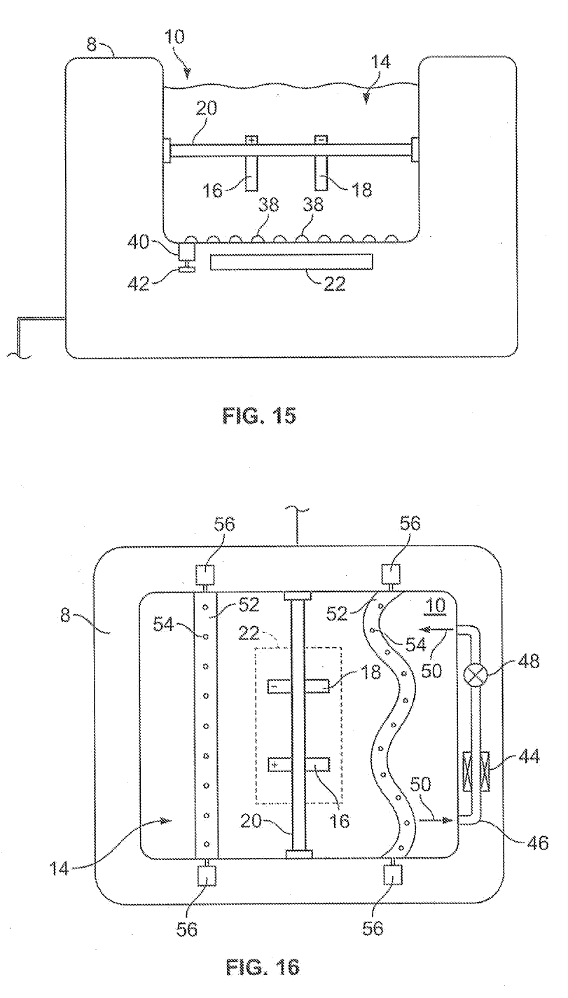

The apparatus may further comprise various mechanical adaptations as shown in FIG. 15-16. For example, the housing 8 may comprise a medium retaining wall for containing the electrically conductive medium 14 and a floor surface. In one variation, the floor may contain a plurality of protrusions 38 while in another variation, the floor may be smooth. Additionally and/or alternatively, the apparatus may have at least one vibrating mechanism to vibrate the floor surface to further enhance a massaging effect on the user's body. The vibration mechanism in one variation may comprise a motor 40 having an output shaft and a rotating member 42 eccentrically fastened to the output shaft with the rotating member 42 coupled to the housing 8 such that when the motor 40 is actuated, rotating member 42 may be urged in an off-axis rotation to impart a vibrational motion to the housing 8.

Another variation may include at least one heating element 44 in thermal conductive contact with the electrically conductive medium 14 to further enhance a massaging effect on the user's body. The heating element 44 may comprise of a fluid channel 46 in communication with the reservoir 10 and a pump 48 to circulate the electrically conductive medium 14 through the fluid channel 46 and into thermal contact with the heating element, as shown by arrows 50. Additionally and/or alternatively, the apparatus may further comprise of a bubble generator within the housing 8 having one or more channels 52 defined along the housing. The channels 52 define a plurality of openings 54 along a length of the channel 52 and is connected to at least one pump 56 which may urge air through the one or more channels 52. In this manner, bubbles may be formed and vented into the medium 14 through the plurality of openings 54. The one or more of the channels may be linear or non-linear in configuration while in other variations, two or more channels may each be linear and non-linear.

While illustrative examples are described above, it will be apparent to one skilled in the art that various changes and modifications may be made therein. Moreover, various apparatus or methods described above are also intended to be utilized in combination with one another, as practicable. The appended claims are intended to cover all such changers and modifications that fall within the true spirit and scope of the invention.

What is claimed is:

1. An apparatus for effecting therapy on a portion of a patient's body comprising:
a housing defining a reservoir for receiving a portion of the body at least partially within an electrically conductive medium, wherein a portion of the reservoir contains at least one positively charged electrode and at least one negatively charged electrode;
a magnet in proximity to the electrodes; and
a controller unit in communication with the electrodes and/or magnet.

2. The apparatus of claim 1, wherein the electrically conductive medium is a fluid.

3. The apparatus of claim 2, wherein the electrically conductive medium is water.

4. The apparatus of claim 1, wherein the electrodes each comprise a conductive metal plate.

5. The apparatus of claim 4, wherein the conductive metal plates are positioned parallel relative to one another.

6. The apparatus of claim 1, further comprising a retaining member positioned to secure the electrodes to the housing.

7. The apparatus of claim 6, wherein the retaining member is securable to the housing and is in electrical communication with the electrodes.

8. The apparatus of claim 1, further comprising a power source in electrical communication with the electrodes.

9. The apparatus of claim 1, wherein the controller unit is configured to regulate an electric current to the electrodes such that an electric field is induced through the electrically conductive medium in the presence of a magnetic field generated via the magnet.

10. The apparatus of claim 9, wherein the controller unit comprises a current limiting switch.

11. The apparatus of claim 9, wherein the controller unit comprises a timer.

12. The apparatus of claim 9, wherein the electric field is configured to be static.

13. The apparatus of claim 9, wherein the electric field is configured to fluctuate.

14. The apparatus of claim 1, further comprising a biofeedback unit adapted to detect a physiological parameter from the patient's body and which is in communication with the controller unit.

15. The apparatus of claim 14, wherein the biofeedback unit is adapted to detect a heartbeat.

16. The apparatus of claim 14, further comprising a user interface to display the physiological parameter.

17. The apparatus of claim 15, wherein the controller unit is adapted to regulate an electric current through the electrodes such that an electric field generated via the electrodes through the conductive medium is induced between heartbeats.

18. The apparatus of claim 1, wherein the magnet is an electromagnet adapted for generating an electromagnetic field.

19. The apparatus of claim 18, wherein the electromagnet is in electrical communication with the controller unit.

20. The apparatus of claim 19, wherein a magnetic field generated via the electromagnet is static.

21. The apparatus of claim 19, wherein a magnetic field generated via the electromagnet fluctuates.

22. The apparatus of claim 1, wherein the housing comprises a medium retaining wall for containing the conductive medium and a floor surface.

23. The apparatus of claim 22 wherein the floor surface is smooth.

24. The apparatus of claim 22, wherein the floor comprises a plurality of protrusions.

25. The apparatus of claim 22, further comprising a vibrating mechanism adapted to vibrate the floor surface.

26. The apparatus of claim 25, wherein the vibrating mechanism comprises:
a motor having an output shaft; and
a rotating member coupled to the housing wherein the rotating member is eccentrically fastened to the output shaft.

27. The apparatus of claim 1, further comprising a heating element in thermally conductive contact with the conductive medium.

28. The apparatus of claim 27, wherein the heating element further comprises:
a fluid channel in fluid communication with the reservoir; and
a pump adapted to circulate the electrically conductive medium through the fluid channel and into thermal contact with the heating element.

29. The apparatus of claim 1, further comprising a bubble generator within the housing.

30. The apparatus of claim 29, wherein the bubble generator comprises:
one or more channels defined along the housing, the one or more channels defining a plurality of openings along a length of the channel; and
a pump adapted to urge air through the one or more channels whereby bubbles are formed and vented through the plurality of openings into the conductive medium.

31. The apparatus of claim 30, wherein the one or more channels are linear.

32. The apparatus of claim 30, wherein the one or more channels are non-linear.

33. A method for improving circulation within a portion of a patient's body comprising:
inducing an electric field through an electrically conductive medium while the portion of the body is at least partially immersed therein;
inducing a magnetic field in proximity to the induced electric field; and
measuring a physiological parameter of the body while the portion is subjected to the electric and magnetic fields.

34. The method of claim 33, wherein inducing an electric field comprises passing an electrical current from a positively charged electrode to a negatively charged electrode at least partially immersed in the conductive medium.

35. The method of claim 34 wherein the electric field is induced perpendicularly relative to the magnetic field.

36. The method of claim 34, further comprising regulating the electric field and/or magnetic field via controller unit.

37. The method of claim 34, wherein inducing an electric field comprises inducing a static electric field.

38. The method of claim 34, wherein inducing an electric field comprises inducing a fluctuating electric field.

39. The method of claim 37, wherein inducing a static electric field comprises pulsing an electric current at a constant amplitude and frequency.

40. The method of claim 38, wherein inducing a fluctuating electric field comprises pulsing an electric current at a varied amplitude and frequency.

41. The method of claim 33, wherein measuring a physiological parameter comprises detecting a heartbeat.

42. The method of claim 41, further comprising regulating an electric current to pulse in a corresponding manner between detected heartbeats.

43. The method of claim 42, wherein the electric current is pulsed between heartbeats at a constant amplitude and a constant frequency.

44. The method of claim 42, wherein the electric current is pulsed between heartbeats at a varied amplitude and a varied frequency.

45. The method of claim 42, wherein the electric current is pulsed at 72 pulses per minute if measuring a physiological parameter fails to detect a heartbeat.

46. The method of claim 41, wherein the physiological parameter is detected via a biofeedback unit.

47. A method for improving circulation within a portion of a patient's body comprising:
inducing an electric field through an electrically conductive medium while the portion of the body is at least partially immersed therein;
inducing an electromagnetic field in proximity to the induced electric field; and
measuring a physiological parameter of the body while the portion is subjected to the electric and electromagnetic fields.

48. The method of claim 47, wherein inducing an electric field comprises passing an electrical current from a positively charged electrode to a negatively charged electrode at least partially immersed in the conductive medium.

49. The method of claim 48, wherein the electromagnetic field is induced perpendicularly relative to the electric field.

50. The method of claim 47, further comprising regulating the electromagnetic field via a controller unit.

51. The method of claim 48, wherein inducing an electric field comprises inducing a static electric field.

52. The method of claim 50, wherein inducing an electric field comprises inducing a fluctuating electric field.

53. The method of claim 51, wherein inducing a static electric field comprises pulsing an electric current at a constant amplitude and frequency.

54. The method of claim 52, wherein inducing a fluctuating electric field comprises pulsing an electric current at a varied amplitude and frequency.

55. The method of claim 47, wherein measuring a physiological parameter comprises detecting a heartbeat.

56. The method of claim 55, further comprising regulating an electric current to pulse in a corresponding manner between detected heartbeats.

57. The method of claim 56, wherein the electric current is pulsed between heartbeats at a constant amplitude and a constant frequency.

58. The method of claim 56, wherein the electric current is pulsed between heartbeats at a varied amplitude and a varied frequency.

59. The method of claim 56, wherein the electric current is pulsed at 72 pulses per minute if measuring a physiological parameter fails to detect a heartbeat.

60. The method of claim 54, wherein the physiological parameter is detected via a biofeedback unit.
